SEQUENCE LISTING (23 sequences)

SEQ ID NO: 1 - DNA, 564 bp, Homo sapiens
  source 1..564: mol_type = genomic DNA
cctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcc

SEQ ID NO: 2 - DNA, 1017 bp, Mycobacterium tuberculosis
  source 1..1017: mol_type = genomic DNA
atgcagcttgttgacagggttcgtggcgccgtcacgggtatgtcgcgtcgactcgtggtcggggccgtcggcgcggccctagtgtcgggtctggtcggcgccgtcggtggcacggcgaccgcgggggcattttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggcgcgcagctcaacgctatgaagcccgacctgcaacgggcactgggtgccacgcccaacaccgggcccgcgccccagggcgcctag

SEQ ID NO: 3 - DNA, 523 bp, synthetic construct
  source 1..523: mol_type = other DNA
atgctcgtgaacagccgggaggataacagacagtacggctttgtgaaccacagcttcactcttgtggcgacggtgaccatccaccaggttgcgaaggggagcactcctctgctgggggcgggtctggatgcacccgtcagcacgaacttcattggtcgacaatgggaggatgcgtaccgctgcgtcaacgcaagcacggcaaatgcggagagggttccgaacggtttgaagtttgcgggggttggcggaggggcgctttggccggtgagccagcaggggcagaatcaacggtatcactttgcaaaccacgcgttcacgctggtggcgtcggtgacgattcacgaggttccgagcgtcgcgagtcctttgctgggtgcgagcctggactcttctggtggcaaaaaactcctggggctctcgtacgacgagaagcaccagtggcagccaatatacggatcaacgccggtgacgccgaccggatcgtgggagatgggtaagaggtaccacgtggttctttctagaa

SEQ ID NO: 4 - DNA, 639 bp, Foot-and-mouth disease virus
  source 1..639: mol_type = other DNA
accaccgctactggggaatcagcagaccctgtcaccaccactgtagagaactacggcggtgagacacaagttcagagacgccaccacaccgacgttggcttcatcatggacagatttgtgaaaataaacagcccaaaatccacccatgtcattgacctcatgcaaacccaccaacacggtctagtgggtgcgctgctgcgtgcggcgacctactacttctcagatctggaaattgttgtgcggcacgacggtaacctaacttgggtgcccaatggtgctcccgtgtcagccttgtccaacaccagcaaccccaccgcctacaacaaggcaccgttcacgagacttgccctcccctacaccgcgccacaccgcgtgttggcgactgtgtacaacgggacgagcaagtacactgtgagtgggttaagcagacgaggcgacttgggttccctcgcggcacgagtcgcgaaggcacttcctgcttctttcaactacggtgcaatcaaggccgtcaacgtgcacgagcttctcgtgcgcatgaaacgggccgaactctactgccctagaccactgttggcactagaggtgatctcacaagacagacacaaacagaagatcattgcacccgaaaaacagcttttg

SEQ ID NO: 5 - DNA, 551 bp, Foot-and-mouth disease virus
  source 1..551: mol_type = other DNA
ctcaccactcgtaatgggcacaccacctccacgacccagtccagcgtaggcgttacatacgggtactccacaacagaggaccacgttgctggacccaacacatcaggtttggagacacgagtggtacaggcagagagattctacaaaaagtttttgtttgattggacaacggacaagccttttggacacctgcacaaactggagttgcccaccgaccactacggtgttttcggacacttggtggactcatacgcctacatgaggaacggttgggacgttgaggtgtctgctgttggcaaccagttcaacggcggatgcctcctagtggccatggtacccgaatggaaagagtttgaaacgcgggagaagtaccagctcacgcttttcccgcaccagttcattagccccagaaccaacatgactgcccacatcacggttccttaccttggtgtgaatagatatgatcagtacagaaaacacaaaccctggacactggttgtcatggtcgtgtccccgctcacggccaacgccacgagcgcggcacagatcaa

SEQ ID NO: 6 - DNA, 1323 bp, Vesicular stomatitis Indiana virus
  source 1..1323: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttatttttt

SEQ ID NO: 7 - protein, 542 aa, synthetic construct
MPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGGGSMALVPVGMAPRQMRVNRCIFASIVSFDACITYKSPCSPDAYHDDGWFICNNHLIKRFKMSKMVLPIFDEDDNQFKMTIARHLVGNKERGIKRILIPSATNYQDVFNLNSMMQAEQLIFHLIYNNENAVNTICDNLKYTEGFTSNTQRVIHSVYATTKSILDTTNPNTFCSRVSRDELRFFDVTNARALRGGAGDQLFNNYSGFLQNLIRRAVAPEYLQIDTEELRFRNCATCIIDETGLVASVPDGPELYNPIRSSDIMRSQPNRLQIRNVLKFEGDTRELDRTLSGYEEYPTYVPLFLGYQIINSENNFLRNDFIPRANPNATLGGGAVAGPAPGVAGEAGGGIAV

SEQ ID NO: 8 - protein, 690 aa, synthetic construct
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGAGGGGSMALVPVGMAPRQMRVNRCIFASIVSFDACITYKSPCSPDAYHDDGWFICNNHLIKRFKMSKMVLPIFDEDDNQFKMTIARHLVGNKERGIKRILIPSATNYQDVFNLNSMMQAEQLIFHLIYNNENAVNTICDNLKYTEGFTSNTQRVIHSVYATTKSILDTTNPNTFCSRVSRDELRFFDVTNARALRGGAGDQLFNNYSGFLQNLIRRAVAPEYLQIDTEELRFRNCATCIIDETGLVASVPDGPELYNPIRSSDIMRSQPNRLQIRNVLKFEGDTRELDRTLSGYEEYPTYVPLFLGYQIINSENNFLRNDFIPRANPNATLGGGAVAGPAPGVAGEAGGGIAV

SEQ ID NO: 9 - protein, 526 aa, synthetic construct
MLVNSREDNRQYGFVNHSFTLVATVTIHQVAKGSTPLLGAGLDAPVSTNFIGRQWEDAYRCVNASTANAERVPNGLKFAGVGGGALWPVSQQGQNQRYHFANHAFTLVASVTIHEVPSVASPLLGASLDSSGGKKLLGLSYDEKHQWQPIYGSTPVTPTGSWEMGKRYHVVLSRGGGGSMALVPVGMAPRQMRVNRCIFASIVSFDACITYKSPCSPDAYHDDGWFICNNHLIKRFKMSKMVLPIFDEDDNQFKMTIARHLVGNKERGIKRILIPSATNYQDVFNLNSMMQAEQLIFHLIYNNENAVNTICDNLKYTEGFTSNTQRVIHSVYATTKSILDTTNPNTFCSRVSRDELRFFDVTNARALRGGAGDQLFNNYSGFLQNLIRRAVAPEYLQIDTEELRFRNCATCIIDETGLVASVPDGPELYNPIRSSDIMRSQPNRLQIRNVLKFEGDTRELDRTLSGYEEYPTYVPLFLGYQIINSENNFLRNDFIPRANPNATLGGGAVAGPAPGVAGEAGGGIAV

SEQ ID NO: 10 - protein, 565 aa, synthetic construct
TTATGESADPVTTTVENYGGETQVQRRHHTDVGFIMDRFVKINSPKSTHVIDLMQTHQHGLVGALLRAATYYFSDLEIVVRHDGNLTWVPNGAPVSALSNTSNPTAYNKAPFTRLALPYTAPHRVLATVYNGTSKYTVSGLSRRGDLGSLAARVAKALPASFNYGAIKAVNVHELLVRMKRAELYCPRPLLALEVISQDRHKQKIIAPEKQLLGGGGSMALVPVGMAPRQMRVNRCIFASIVSFDACITYKSPCSPDAYHDDGWFICNNHLIKRFKMSKMVLPIFDEDDNQFKMTIARHLVGNKERGIKRILIPSATNYQDVFNLNSMMQAEQLIFHLIYNNENAVNTICDNLKYTEGFTSNTQRVIHSVYATTKSILDTTNPNTFCSRVSRDELRFFDVTNARALRGGAGDQLFNNYSGFLQNLIRRAVAPEYLQIDTEELRFRNCATCIIDETGLVASVPDGPELYNPIRSSDIMRSQPNRLQIRNVLKFEGDTRELDRTLSGYEEYPTYVPLFLGYQIINSENNFLRNDFIPRANPNATLGGGAVAGPAPGVAGEAGGGIAV

SEQ ID NO: 11 - protein, 516 aa, synthetic construct
TTQSSVGVTYGYSTTEDHVAGPNTSGLETRVVQAERFYKKFLFDWTTDKPFGHLHKLELPTDHYGVFGHLVDSYAYMRNGWDVEVSAVGNQFNGGCLLVAMVPEWKEFETREKYQLTLFPHQFISPRTNMTAHITVPYLGVNRYDQYRKHKPWTLVVMVVSPLTGGGGSMALVPVGMAPRQMRVNRCIFASIVSFDACITYKSPCSPDAYHDDGWFICNNHLIKRFKMSKMVLPIFDEDDNQFKMTIARHLVGNKERGIKRILIPSATNYQDVFNLNSMMQAEQLIFHLIYNNENAVNTICDNLKYTEGFTSNTQRVIHSVYATTKSILDTTNPNTFCSRVSRDELRFFDVTNARALRGGAGDQLFNNYSGFLQNLIRRAVAPEYLQIDTEELRFRNCATCIIDETGLVASVPDGPELYNPIRSSDIMRSQPNRLQIRNVLKFEGDTRELDRTLSGYEEYPTYVPLFLGYQIINSENNFLRNDFIPRANPNATLGGGAVAGPAPGVAGEAGGGIAV

SEQ ID NO: 12 - DNA, 28 bp, synthetic construct
  source 1..28: mol_type = other DNA
acccgggaaagttcaccatagtttttcc

SEQ ID NO: 13 - DNA, 29 bp, synthetic construct
  source 1..29: mol_type = other DNA
gactgcagaaaaaataaactctcatcatc

SEQ ID NO: 14 - DNA, 28 bp, synthetic construct
  source 1..28: mol_type = other DNA
aggatccaatatgcctttcagagtgact

SEQ ID NO: 15 - DNA, 25 bp, synthetic construct
  source 1..25: mol_type = other DNA
atctagaaggggaaacacatctgcc

SEQ ID NO: 16 - DNA, 25 bp, synthetic construct
  source 1..25: mol_type = other DNA
aggatccaatatgcagcttgttgac

SEQ ID NO: 17 - DNA, 22 bp, synthetic construct
  source 1..22: mol_type = other DNA
atctagactaggcgccctgggg

SEQ ID NO: 18 - DNA, 27 bp, synthetic construct
  source 1..27: mol_type = other DNA
actgcagcgaccaccgctactggggaa

SEQ ID NO: 19 - DNA, 27 bp, synthetic construct
  source 1..27: mol_type = other DNA
actgcagcgctgtttttcgggtgcaat

SEQ ID NO: 20 - DNA, 34 bp, synthetic construct
  source 1..34: mol_type = other DNA
actcgagaatatggccgacaagaagacggaggag

SEQ ID NO: 21 - DNA, 25 bp, synthetic construct
  source 1..25: mol_type = other DNA
atctagactctttcgaggggagctc

SEQ ID NO: 22 - DNA, 33 bp, synthetic construct
  source 1..33: mol_type = other DNA
aagatctaccatgctcgtgaacagccgggagga

SEQ ID NO: 23 - DNA, 27 bp, synthetic construct
  source 1..27: mol_type = other DNA
atctagaaagaaccacgtggtacctac

(19) United States
(12) Patent Application Publication
(10) Pub. No.: US 2025/0000961 A1
(43) Pub. Date: Jan. 2, 2025

(54) RECOMBINANT BACULOVIRUSES, VACCINE COMPOSITIONS THAT COMPRISE IT, AND METHODS FOR INDUCING AN IMMUNE RESPONSE

(71) Applicants: Instituto Nacional de Tecnologia Agropecuaria, C.A.B.A. (AR); Universidad Nacional de Cordoba, Cordoba (AR)

(72) Inventors: Maria Paula Molinari, Buenos Aires (AR); Oscar Alberto Taboga, Buenos Aires (AR); Victor Gabriel Moron, Cordoba (AR); Maria Ines Crespo, Cordoba Capital (AR)

(73) Assignees: Instituto Nacional de Tecnologia Agropecuaria, C.A.B.A. (AR); Universidad Nacional de Cordoba, Cordoba (AR)

(21) Appl. No.: 18/658,166

(22) Filed: May 8, 2024

Related U.S. Application Data
(60) Provisional application No. 63/464,988, filed on May 9, 2023.

(51) Int. Cl. A61K 39/04; A61K 39/00; A61K 39/005; A61K 39/135; A61P 37/04; C12N 7/00

(52) U.S. Cl. CPC A61K 39/04; A61K 39/005; A61K 39/135; A61P 37/04; C12N 7/00; A61K 2039/5256; A61K 2039/6075; C12N 2710/14021; C12N 2710/14022; C12N 2760/20222; C12N 2770/32122; C12N 2770/32134

(57) ABSTRACT

A recombinant baculovirus having a) a nucleotide sequence encoding a fusion protein, wherein said fusion protein includes an antigen fused to a baculovirus capsid peptide operatively linked to a first promoter and b) a nucleotide sequence encoding a lipid viral envelope protein bound to a second promoter.

### CROSS REFERENCE TO RELATED APPLICATION

This application claim priority over U.S. Provisional application No. 63/464,988 filed May 9, 2023, the content of which is incorporated herein by reference.

### SEQUENCE LISTING

The present application is being filed along with a Sequence Listing in electronic format. The Sequence Listing is provided as a file entitled INTAFinal.xml, created May 5, 2024, which is 28,477 bytes in size. The information in the electronic format of the Sequence Listing is incorporated herein by reference in its entirety.

### BACKGROUND

Despite the success achieved by conventional vaccines in preventing various infectious diseases, there is an urgent need for innovative vaccine platforms capable of addressing emerging pathogenic threats and overcoming the shortcomings of traditional methods. One of these shortcomings is that they need adjuvants or multiple doses to achieve the desired immune responses, which may lead to logistical challenges and affect vaccine efficiency.

Baculoviruses belong to a family of enveloped, rod-shaped viruses with a circular double-stranded DNA genome (80-200 kb). Baculoviruses infect some insects, but not mammals (Blissard, Cytotechnology 20:73-93, 1996). The californica multiple nucleopolyhedrovirus (AcMNPV) is the best studied and most used for protein expression because the polyhedrin (PH) and p10 promoters are efficacious promoters (McMichael et al., N Engl J Med 309:13-17, 1983).

The use of baculovirus-based vaccine vectors represents a promising solution to health challenges. These viruses, which are non-pathogenic to humans, have a demonstrated safety record in various biotechnological applications. Furthermore, their ability to include large gene fragments and efficiently transport these foreign genes to target cells makes them ideal candidates for vaccine development.

Due to their low cytotoxicity and absence of preexisting antibodies against AcMNPV, these viruses have emerged as strong candidates for the design of vaccine vectors since it is possible to present immunogens or heterologous peptides in different structures such as the AcMNPV envelope through fusion with a viral membrane protein (Oker-Blom et al., Brief Funct Genomic Proteomic 2:244-253, 2003).

Baculoviruses have also been used for producing particulate immunogens for vaccines against HIV, HPV and influenza (Gheysen et al., Cell 59:103-112, 1989; Kirnbauer et al., J Virol 67:6929-6936), 1993; Latham et al., J Virol 75:6154-6165, 2001).

There are also other technologies that allow modification of cell membranes and enveloped viruses with heterologous proteins which confer new properties such as increasing capability to enter into more cells by endocytosis. The human syncytial respiratory virus G gene expresses both a type II membrane-anchored glycoprotein and a soluble protein. The G protein is strongly O-glycosylated and shows significant structural similarities to mucinous proteins. The G protein was initially characterized for providing a binding function, and, since then, domains in G which bind sulfated glycosaminoglycans on the cell surface in vitro have been identified.

In most infectious diseases, antibody response is not sufficient to eliminate pathogens. Rather, a strong CD8+T-mediated immune response is also required. In the case of tumor diseases, CD8+ T cell-mediated immunity is also essential to generate an efficient antitumoral immunity.

In view of the continuous global health challenges posed by emerging infectious diseases and the persistent threats of pandemics, development of novel vaccine platforms, such as baculovirus-based vectors, is imperative. By leveraging the unique properties of these vectors and advances in biotechnology, researchers may advance toward the creation of next generation vaccines offering improved safety, efficiency, and scalability, thus promoting the goal of global health safety.

### BRIEF DESCRIPTION OF THE INVENTION

A recombinant baculovirus comprising a) a nucleotide sequence encoding a fusion protein, consisting of an antigen fused to a baculovirus capsid peptide operatively linked to a first promoter and b) a nucleotide sequence encoding a lipid viral envelope protein bound to a second promoter is provided. The antigen may be any type of antigen, for example and without limitation: viral antigens, bacterial antigens, tumor antigens or parasite antigens. In a preferred embodiment the baculovirus capsid peptide may be VP39, p6.9, Ac104, Ac144, Ac101, Ac109, Ac142, Ac98 or variants and fragments thereof. In an even more preferred embodiment, the peptide may be VP39 encoded by the nucleotide sequence set forth in SEQ ID No. 6. The expressed fusion protein may comprise the amino acid sequences of SEQ ID No. 7, SEQ ID No.8, SEQ ID No. 9, SEQ ID No. 10 and SEQ ID No. 11.

In a preferred embodiment, the antigen of the fusion protein may be MO5, Mycobacterium tuberculosis Ag85A, foot-and-mouth virus VP1, foot-and-mouth virus VP2, Trypanosoma cruzi ASP2 fused to Trypanosoma cruzi TS or variants and fragments thereof. In another preferred embodiment, the antigen may be the MO5 epitope, Mycobacterium tuberculosis Ag85A encoded by the nucleotide sequence of SEQ ID No. 2, the foot-and-mouth virus VP1 encoded by the nucleotide sequence of SEQ ID No. 4, foot-and-mouth virus VP2 encoded by the nucleotide sequence of SEQ ID No. 5 or Trypanosoma cruzi ASP2 fused to Trypanosoma cruzi TS encoded by the nucleotide sequence of SEQ ID No. 3.

The first and second promoter may be any insect cell promoter, for example, and not limited to, Ppol, P10, Pie1, PTriEx, or these promoters in tandem. In some cases the second promoter may be a promoter which is active in animal cells, for example, PCMV, PCAG, PTriEx, pSV40 or these promoters in tandem.

The lipid viral envelope protein may be the vesicular stomatitis virus (VSV) G protein, or Penetrin-like proteins having a similar molecular structure or topology, for example G (Rhabdovirus), GP (Thogo virus), gB (Herpes virus). In a preferred embodiment, the lipid viral envelope protein is G of the vesicular stomatitis virus and is encoded by the nucleotide sequence set forth in SEQ ID No. 6.

Further described are recombinant baculoviruses comprising a nucleotide sequence encoding a fusion protein, consisting of an antigen fused to a baculovirus capsid peptide operatively linked to a promoter, where the fusion protein comprises the amino acid sequence of SEQ ID No. 7, SEQ ID No. 8, SEQ ID No. 9, SEQ ID No. 10, or SEQ ID No. 11, and wherein the fusion protein is expressed on the capsid. The promoter may be Ppol, P10, Pie1, Ptrix, or these promoters in tandem.

Further described are recombinant baculoviruses comprising a nucleotide sequence encoding the vesicular stomatitis virus G protein operatively linked to a promoter, where the G protein is encoded by the nucleotide sequence of SEQ ID No. 6. The promoter may be Ppol, P10, Pie1, PTriEx, PSV40, PCMV, or PCAG.

A vaccine composition comprising the recombinant baculovirus comprising a) a nucleotide sequence encoding a fusion protein, wherein said fusion protein consists of an antigen fused to a baculovirus capsid peptide operatively linked to a first promoter and b) a nucleotide sequence encoding a lipid viral envelope protein bound to a second promoter, is provided. The antigen may be any type of antigen, for example and without limitations: multi-antigen viral antigens, bacterial antigens, tumor antigens or parasite antigens.

A vaccine composition of a recombinant baculovirus comprising a first baculovirus carrying a nucleotide sequence encoding a fusion protein, wherein said fusion protein consists of an antigen fused to a baculovirus capsid peptide operatively linked to a promoter, wherein the fusion protein comprises the amino acid sequence of SEQ ID No. 7, SEQ ID No. 8, SEQ ID No. 9, SEQ ID No. 10, and SEQ ID No. 11, and wherein the fusion protein is expressed on the capsid, is provided. The promoter may be Ppol, P10, Pie1, PTriEx, or these promoters in tandem; and a second recombinant baculovirus comprising a nucleotide sequence encoding the vesicular stomatitis virus G protein, operatively linked to a promoter, wherein the G protein is encoded by the nucleotide sequence of SEQ ID No.6 and the promoter may be Ppol, P10, Pie1, PTriEx, -PCMV, PSV40, pCAG or these promoters in tandem.

In one embodiment, a method for inducing an immune response comprising administering to a subject a pharmaceutically effective amount, for example, from 1×106 to 2,7×108 CFU of baculovirus, wherein the baculovirus may be a recombinant baculovirus comprising a) a nucleotide sequence encoding a fusion protein, wherein said fusion protein consists of an antigen fused to a baculovirus capsid peptide operatively linked to a first promoter and b) a nucleotide sequence encoding a lipid viral envelope protein bound to a second promoter, is provided. The antigen may be any type of antigen, for example and without limitations viral antigens, bacterial antigens, tumor antigens, -intracellular pathogens, parasite antigens, combined multi-antigens/antigens or fragments of combined antigens.

In another embodiment, a method for inducing an immune response comprising administering to a subject a pharmaceutically effective amount, for example, from 1×106 to 2,7×108 CFU of a first baculovirus carrying a nucleotide sequence encoding a fusion protein, wherein said fusion protein consists of an antigen fused to a baculovirus capsid peptide operatively linked to a promoter, wherein the fusion protein comprises the amino acid sequence of SEQ ID No. 7, SEQ ID No. 8, SEQ ID No. 9, SEQ ID No. 10, or SEQ ID No. 11, and wherein the fusion protein is expressed on the capsid, is provided The promoter may be Ppol, P10, Pie1, PTriEx, or these promoters in tandem; and from 1×106 to 2,7×108 CFU of a second recombinant baculovirus comprising a nucleotide sequence encoding the vesicular stomatitis virus G protein or rabies G virus or Thogovirus GP, or Herpesvirus gB operatively linked to a promoter; in a preferred embodiment the G protein is encoded by the nucleotide sequence of SEQ ID No. 6 and the promoter may be Ppol, P10, Pie1 PTriEx, PCAG, PCMV-PSV40 or these promoters in tandem.

### DESCRIPTION OF THE DRAWINGS

FIG. 1 shows a representation of the AcMNPV: AcMNPV (pol-) recombinant cassettes, virus without recombinant protein; DNA fragments of the antigens OVA (SEQ ID No. 1), Ag85A (SEQ ID No. 2), ASP2TSChagas (SEQ ID No. 3), VP1FMDV (SEQ ID No. 4), and VP2FMDV (SEQ ID No. 5) were cloned into the pFBcap plasmid to generate the recombinant baculoviruses AcMNV-OVAcap, AcMNV-85ATBcap, AcMNV-ASP2TSchagascap, AcMNV-VP1FMDVcap, and AcMNV-VP2FMDVcap. The Ssgp64 sequence is the export signal sequence of the baculoviral GP64 protein. VP39, in all its versions, has a linker sequence (GGGGS) at the N-terminus of VP39 (cap). The nucleotide sequence of the VSV G protein (SEQ ID No.6) is cloned into the pFB plasmid to generate the AcMNPVG virus. Ppol is the polyhedrin promoter of the AcMNPV c virus, PCMV is the promoter cytomegalovirus, p10 is the promoter 10 of the AcMNPV virus.

FIG. 2 shows the expression of fusion proteins in purified recombinant baculoviruses, resolved by SDS-PAGE and detected by Western blot using specific sera or monoclonal antibodies for each one and/or for VP39. For AcMNPV-OVAcap (rabbit anti-OVA polyclonal serum) AcMNPV-VP1FMDVcap or AcMNPV-VP2FMDVcap (anti-FMDV A2001 polyclonal serum). The anti VP39 monoclonal antibody was used for AcMNPV-OVAcap, AcMNPV-VP1FMDVcap, AcMNPV-VP2FMDVcap, AcMNPV-Ag85ATBcap, and AcMNPV-ASP2TSchagascap.

FIG. 3 shows the co-expression of antigens and G protein in the viral particles of the invention. Purified recombinant baculoviruses AcMNPV-OVAcap, AcMNPV-(G) OVAcap, and AcMNPV, were resolved by SDS-PAGE and detected by Western blot using anti-ovoalbumin serum (upper figure) or anti G monoclonal antibodies (lower figure).

FIG. 4: BMDC cells differentiated in the presence of the Flt3-L cytokine were labelled with different antibodies and a viability marker. Then they were analyzed by flow cytometry to evaluate phenotype and frequency of the different DC populations. The analysis was carried out for the population of CD11c+ viable cells.

FIG. 5 shows BMDC cells incubated with different baculoviruses at the specified MOI for 4 hours at 37° C. The cells were then washed and incubated with the B3Z cell line for 18 hours at 37° C. Presentation of the MHCI/OVA257-264 complex and its subsequent activation of T cells was detected by evaluating OD at 595 nm after addition of CPRG, a β-galactosidase enzyme substrate.

FIG. 6 shows BMDC cells incubated with different baculoviruses at the specified MOI for 4 hours at 37° C. The cells were then washed and incubated with the B3Z cell line for 18 hours at 37° C. Presentation of the MHCI/OVA257-264 complex and its subsequent activation of T cells was detected by evaluating OD at 595 nm after addition of CPRG, a β-galactosidase enzyme substrate.

FIG. 7 shows in vivo studies of the cytotoxic response generated by recombinant baculoviruses. C57BL/6 mice of 6 to 8 weeks of age were i.v. immunized with different amounts of each of the viruses. On post-immunization day 7, immunized animals were i.v. administered an equicellular mixture of syngenic splenocytes previously incubated with the OVA257-264 peptide and stained with a high concentration of Cell Proliferation Dye eFluor™ 670 (target cells) and normal splenocytes stained with a low concentration of the same dye (internal control). Spleens of the animals were obtained after twenty-four hours to evaluate the percentage of Cell Proliferation Dye eFluor™ 670-positive cells remaining therein by flow cytometry and the percentage of specific lysis was calculated.

The following equation was used for calculating the percentage of specific lysis: (1−Rcontrol/Rimmunized)×100, where R=(% internal control cells)/(% target cells) for each evaluated mouse.

### DETAILED DESCRIPTION OF THE INVENTION

### Definitions

| | |
|---|---|
| VSV | vesicular stomatitis virus |
| G | VSV virus G protein |
| VP39 | VP39 protein constituent of baculovirus capsid |
| OVA | ovoalbumin protein |
| MO5 | MO5 epitope contained in the OVA sequence |
| FMDV | foot-and-mouth virus |
| Ag85ATB | Ag85A protein corresponding to Mycobacterium |
| | tuberculosis |
| VP1FMDV | VP1 protein of the FMDV virus |
| VP2FMDV | VP2 protein of FMDV |
| ASP2TSchagas | Trypanosoma cruzi ASP2 proteins fused to the |
| | Trypanosoma cruzi TS protein. |
| AcMNPV | Autographa californica multiple nucleopolyhedrovirus |
| AcMNPV (pol-) | Autographa californica multiple nucleopolyhedrovirus |
| | lacking a polyhedron gene. |
| AcMNPV-G | AcMNPV (pol-) containing G protein |
| AcMNPV-OVAcap | AcMNPV (pol-) containing the OVA protein as a fusion |
| | protein to VP39. |
| AcMNPV- | AcMNPV (pol-) containing the TB Ag85A protein as a |
| Ag85ATBcap | fusion protein to VP39. |
| AcMNPV- | AcMNPV (pol-) containing the FMDV VP1 protein as a |
| VP1FMDVcap | fusion protein to VP39. |
| AcMNPV- | AcMNPV (pol-) containing the FMDV VP2 protein as a |
| VP2FMDVcap | fusion protein to VP39. |
| AcMNPV- | AcMNPV (pol-) containing the Trypanosoma cruzi |
| ASP2TSchagascap | ASP2TS protein as a fusion protein to VP39. |
| AcMNPV- | AcMNPV (pol-) containing the OVA protein sequence |
| PCMVOVA | under a mammal promoter (PCMV). |
| AcMNPV-(G) x | AcMNPV (pol-) containing G protein in the specific viral |
| | particle membrane “X”. |
| Example AcMNPV- | AcMNPV-OVAcap containing a G protein on its |
| (G)OVAcap | membrane. |
| Sf9 | A Spodoptera frugiperda species cell line |
| B3Z | hybridoma of specific CD8+ T-cells for OVA 257-264 |
| | residues in the context of MHC class I (H-2Kb). |
| FBS | Fetal bovine serum |
| Pfb | pFastBac 1 commercial plasmid (Invitrogen) |
| pFBcap | pFastBac1 containing the AcMNPV VP39 protein under |
| | the ph promoter |
| pFBdual | pFastBac dual containing promoters P10 and Ppol |
| pFBPCMV | pFastBac1 containing the PCMV promoter, mammal |
| | promoter (cytomegalovirus promoter) |
| pGemT | commercial plasmid |
| CD | dendritic cell |
| BMDCs | dendritic cells derived from bone marrow |
| C57BL/6 | strain C57BL/6 mice |
| Flt3L | Fms like tyrosine kinase 3 cytokine ligand. |
| MHCI | major histocompatibility complex molecule |
| OVA257-264 | ovoalbumin protein epitope |
| CPRG | chlorophenol red β-D-galactopyranoside |
| OIE | BSL-4 containment biosafety level laboratory. |

Recombinant baculoviruses are shown as vaccines inducing a strong CD8+ T-cell immune response. Recombinant baculoviruses are obtained based on engineering of the AcMNPV capsid VP39 protein to transport foreign antigens plus to incorporate VSV vesicular stomatitis G protein of baculovirus, as enhancer element of dendritic cell (DC) activity.

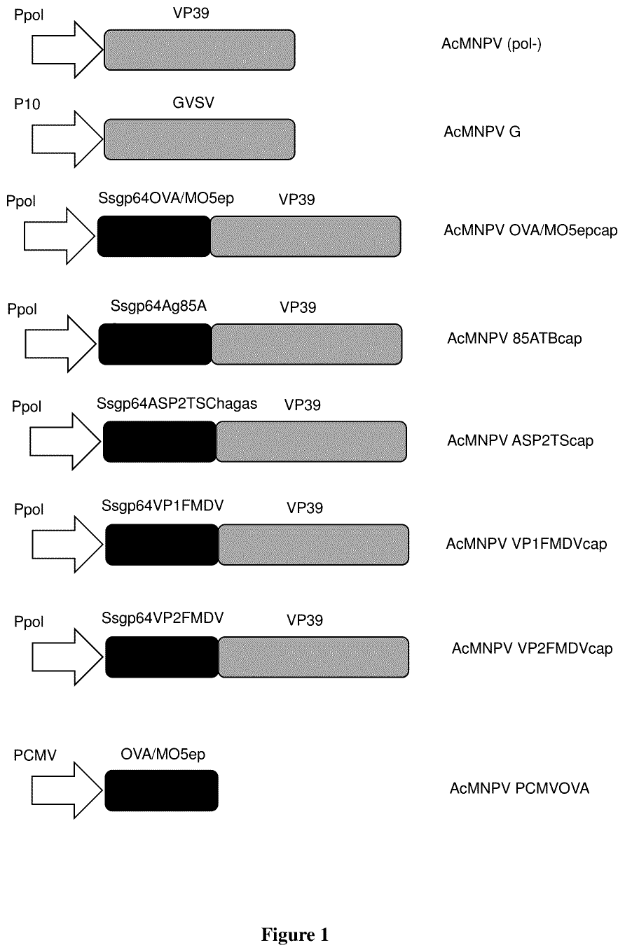

FIG. 1 shows fusion protein constructs for obtaining the AcMNV-OVAcap, AcMNV-85ATBcap, AcMNV-ASP2TSchagascap, AcMNV-VP1FMDVcap, and AcMNV-VP2FMDVcap baculoviruses.

To obtain the baculoviruses of the invention budded recombinant baculovirus AcMNPV-OVAcap virions were pseudotyped as previously described (Molinari, 2011; Molinari, 2018; Tavarone, 2017) with the G protein using the Bac-to-Bac System (Invitrogen, Thermo Fisher Scientific, Buenos Aires, Argentina) to obtain baculoviruses expressing the VSV and OVA G protein as immunogenic antigen or peptide, thereby obtaining the AcMNPV (G) OVAcap virus. Likewise, the baculoviruses AcMNV-(G) 85ATBcap, AcMNV-(G) ASP2TSchagascap, AcMNV-(G) VP1FMDVcap, and AcMNV-(G) VP2FMDVcap were obtained.

For example, the G sequence (SEQ ID No. 6) was amplified and subcloned into the transfer plasmid pFB thus generating the pFBG plasmid with was used for generating the recombinant baculovirus AcMNPVG. On the other hand, the fusion insert to cap OVA, was amplified using the primer described in Example 2 and cloned into the transfer plasmid pFBcap (described in Molinari 2011). The same procedure was repeated for the antigens shown in the sequences of Ag85A1 (SEQ ID No. 2), ASP2TSchagas (SEQ ID No. 3), VP1 (SEQ ID No. 4), VP2 (SEQ ID No. 5), OVA (SEQ ID No. 1). Specific transfer plasmids were generated and used to generate the recombinant baculoviruses AcMNPV-OVAcap, AcMNPV-Ag85ATBcap, AcMNPV-VP1FMDVcap, AcMNPV-VP2FMDVcap, AcMNPV-ASP2TSchagascap. The recombinant baculoviruses were obtained as described in Example 2 and baculoviral titers were calculated as described in Molinari 2011.

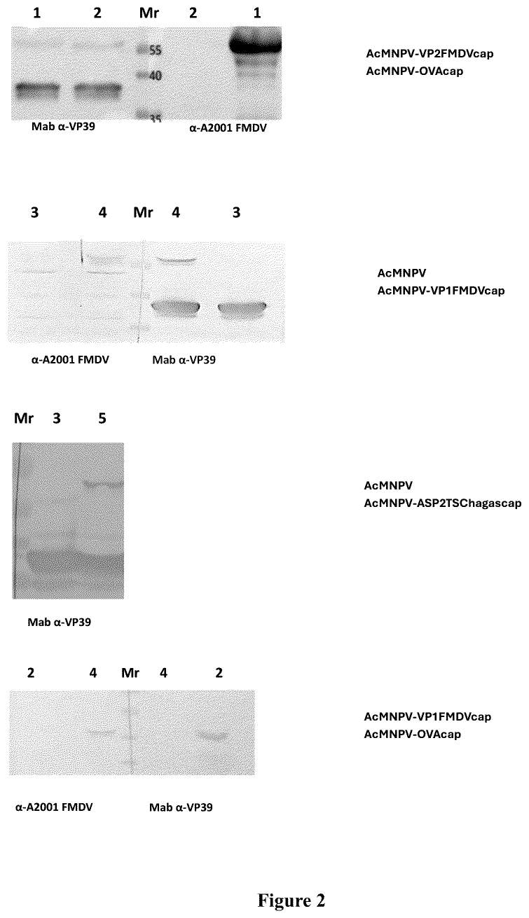

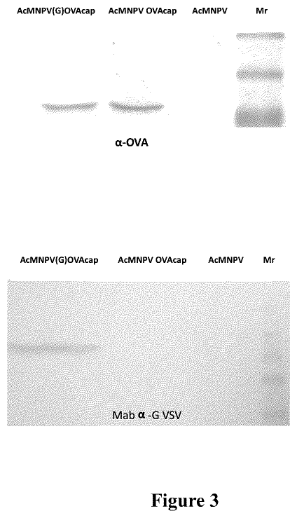

Recombinant baculoviruses were evaluated by collecting Sf9 cells five days after being infected and further purifying the baculoviruses from the supernatants. An ultracentrifugation was performed on a sacarose bed (1.5 h at 80,000×g). Each sample was resolved by 12% SDS polyacrylamide gel electrophoresis and then the proteins were transferred onto nitrocellulose membranes. FIGS. 2 and 3 show characterization of these baculoviruses, which reveal the presence of the G protein, and the antigens OVA, Ag85ATB, VP1FMDV, VP2FMDV, ASP2TSchagas fused to viral protein VP39 in the recombinant baculoviruses; the fusion proteins are shown in FIG. 1.

The recombinant baculoviruses of the invention that carry antigens on the capsid and the G protein in the membrane were obtained as described in example 3 and characterized as described in example 4. Baculoviruses AcMNPV-OVAcap, AcMNPV-PCMVOVA, AcMNPV-Ag85ATBcap, AcMNPV-VP1FMDVcap, AcMNPV-VP2FMDVcap, AcMNPV-ASP2TSchagascap @ and baculoviruses with the following phenotypes AcMNPV-(G) OVApcap, AcMNPV-(G) Ag85ATBcap, AcMNPV-(G) VP1FMDVcap, AcMNPV-(G) VP2FMDVcap, AcMNPV-(G) PCMVOVA and AcMNPV-(G) ASP2Tschagascap were obtained.

Next, the enhancing effect of adding a G protein to the baculovirus membrane on the ability of DCs to activate a cytotoxic T cell mediated immune response (LTCD8) was evaluated. For the in vitro experiments differentiated BMDCs with Flt3-L were used.

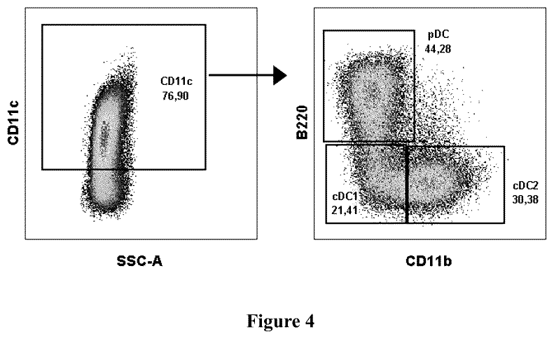

Based on the presence of surface markers and functional analysis, splenic DCs may be grouped as conventional DC1s (cDC1) and DC2s (cDC2), and plasmacytoid DCs (pDC). Differentiation of BMDCs in the presence of the Flt3-L cytokine results in DCs with a phenotype which is similar to the three subpopulations found in the spleen. Once differentiated according to the methodology described in Example 5, a flow cytometry was carried out to evaluate the phenotype and frequency of each of the DC subpopulations thereby obtaining about 80% CD11c+ cells, having the following distribution: 44.3% pDCs (B220+CD11b−), 21.4% cDC2 (B220−CD11b−), 30.4% cDC1 (B220−CD11b+), as shown in FIG. 4. Importantly, said phenotype and frequency were maintained in all experiments with similar values.

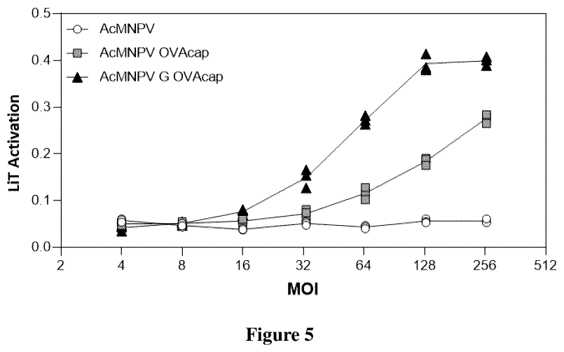

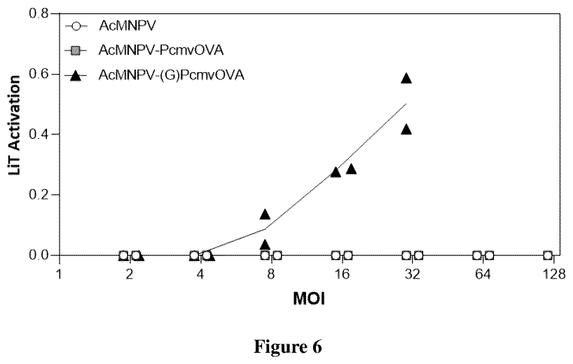

Subsequently an in vitro antigen presentation assay was carried out using the B3Z T cell line whose TCR is specific for the MHC I-presented OVA antigen, see Example 6. To this end, BMDCs were incubated with different baculoviruses expressing OVA on the capsid as an antigen; then the ability of these DCs to activate CD8+ T-cells was evaluated using a colorimetric assay. FIG. 5 shows activation of CD8+ T-cells when DCs were incubated with the baculovirus AcMNPV, AcMNPV-OVAcap and AcMNPV-(G) OVAcap, while FIG. 6 shows the results when the baculoviruses AcMNPV, AcMNPV-PCMVOVA and AcMNPV-(G) PCMVOVA were used.

The results clearly show a greater antigenic presentation when the G protein is in the membrane and the antigen on the baculoviral capsid, being an integral and joint part of the particle baculoviral (AcMNPV (G) OVAcap) with respect to the same baculovirus in the absence of the G protein (AcMNPVOVAcap) in the range of MOI conditions of 16 to 260 (FIG. 6). Similarly, it is noted that the presence of the G protein in the baculovirus membrane in case of the recombinant baculovirus AcMNVPV-PCMVOVA which makes it a adjuvant vector capable stimulating antigenic presentation in DCs (FIG. 6).

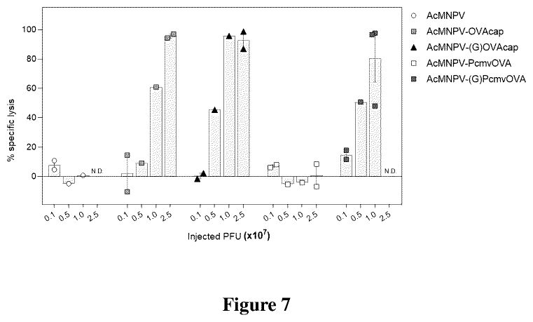

Subsequently, it was evaluated if the recombinant baculovirus of the invention enhances the ability of generating an in vivo CD8+ T-cell cytotoxic response. Therefore C57BL/6 mice were i.v. immunized with different doses of each of the recombinant baculoviruses in a range of from 1×106 to 2.5×107 PFU/animal. As a control, mice were injected with the AcMNPV baculovirus or with physiological solution. After seven days, specific lysis levels were measured in each animal using an in vivo cytotoxicity assay described in Example 7. The use of different doses allowed for determining a dose-dependent effect. According to the results observed in the in vitro antigen presentation assays, it was noted that the recombinant baculoviruses AcMNPV-OVAcap, AcMNPV-(G) OVAcap, and AcMNVPV-(G) PCMVOVA have the ability to activate a CD8+T lymphocyte-mediated cytotoxic response. When compared to the baculoviruses AcMNPV-OVAcap, AcMNPV-(G) PCMVOVA and AcMNPV-(G) OVAcap, it was noted that the presence of the G protein determined higher lysis percentages with a lower amount of viral load in the immunization. This result is clearly observed when comparing the specific lysis percentages obtained in animals injected with 0.5×107 and 1×107 PFU. The addition of the G protein on the membrane of the baculoviruses transforms them into a potent immunogenic vector. (See FIG. 7).

Thus, the recombinant baculoviruses of the invention comprising the G protein promote much more robust antigen specific CD8+ T-cell responses than in the absence of the G protein, both in baculovirus carrying a heterologous antigen fused to VP39 as in baculoviruses expressing the de novo antigen under regulation of a promoter of mammal cells (CMV).

Subsequently, the effect on relevant pathologies was evaluated, such as the foot-and-mouth virus and Mycobacterium tuberculosis antigens.

The effect of foot-and-mouth virus VP1 or VP2 antigens, Mycobacterium tuberculosis Ag85A antigen or MO5ep antigen carried on the capsid on the protection against a lethal challenge with the foot-and-mouth virus or with Mycobacterium tuberculosis or a melanoma model (MO5) murine models was studied.

To that end, mice were immunized with one or two doses of the recombinant baculoviruses AcMNPV-(G) VP1FMDVcap, AcMNPV-(G) VP2FMDVcap, and AcMNPV-(G) Ag85ATBcap; and 7 days after the last dose of recombinant baculovirus they were challenged with FMDV or with MTB, as appropriate. The results showed that the immunization generated an immune response capable of protecting and extending survival of 70-100% mice with a dose of 2×108 PFU per mouse compared with the control. In a similar scheme with a lower dose (0.75×108 PFU) survival was also extended in 40% mice compared to mice that received the control AcMNPV.

On the other hand, immunization with AcMNPV-(G) Ag85ATBcap generated an immune response which decreased M. tuberculosis count in lung 30 days post-challenge, when compared to mice that received control baculovirus and to those which did not receive immunogens.

Finally, a preventive treatment with a dose of 2,7×108 AcMNPV-C) OVA/MO5epcap prevented tumor development in all the animals with respect to 100% death (day 40 post-challenge) in the control group.

The described results clearly demonstrate robustness and immunogenic strength of the antigenic heterologous antigen expression system on the capsid (“capsid display”) plus expression of the VSV G protein in the baculoviral envelope

Baculoviruses thus constructed have the ability of vectorizing heterologous antigens toward dendritic cells. In addition, they provide dendritic cells with strong stimulatory signals that allow for generating a vigorous CD8+ T-cell-mediated cytotoxic response against said antigens.

The results show the important potential of this system to induce protective and therapeutic immune responses against a large series of infectious and neoplastic pathologies wherein the CD8+ T-cell-mediated immune response is essential. The baculoviruses of the invention are very efficient at inducing protection in murine models against viral infections (FMDV, African swine fever, rabies) and parasitic infections (chagas) as well as melanomas.

The baculoviruses described herein express a fusion protein on the capsid comprising an antigen and a specific protein of baculoviral capsids. For example, the fusion protein may be the one shown in the amino acid sequences SEQ ID No. 9, SEQ ID No. 10, SEQ ID No. 11, SEQ ID No. 12 or SEQ ID No. 13. Furthermore, the baculovirus expresses in its membrane a G protein encoded by the nucleotide sequence of SEQ ID No. 6. These baculovirus increase immune response, particularly CD8+ T-cells specific for the antigen and stimulate antigenic presentation in DCs.

It is also shown that it is possible to increase the immune response by applying two different types of baculovirus, one that expresses the fusion protein on the capsid and another baculovirus that expresses the G protein in the membrane, where in the case of the G protein, it may be expressed under a mammalian cell promoter.

### EXAMPLES

### Example 1: Virus, Cell Lines and Culture Media

Baculovirus Autographa californica multiple nucleopolyhedrovirus (AcMNPV) was used and recombinant viruses on AcMNPV (pol-) lacking the polyhedrin protein were produced. The baculoviruses AcMNPV-G, AcMNPV-OVA/MO5epcap, AcMNPV-Ag85ATBcap, AcMNPV-VP1FMDVcap, AcMNPV-VP2FMDVcap, and AcMNPV-ASP2TSchagascap were produced. In addition, the AcMNPV-PCMVOVA baculovirus was produced.

The Sf9 lepidopteran cell line (ATCC CRL-1711), derived from the Spodoptera frugiperda IPLB-Sf-21-AE parental line, was used for propagating the AcMNPV virus. These cells were grown as a monolayer at 27° C. in 75 cm2 Cellstar flasks (Greiner Bio-One) using Grace's medium (ThermoFisher) with 10% fetal bovine serum (Internegocios-SA) and an antibiotic-antimycotic solution (10,000 U/mL penicillin, 10 mg/ml streptomycin, 25 μg/mL amphotericin B, Gibco-BRL) at 27° C.

For the titration of all the recombinant AcMNPVs, the Sf9-pXXLGFP cell line, expressing GFP under the polyhedrin promoter (infection-inducible), was used. It was kept under selective pressure of the blasticidin antibiotic (Life Technologies), at a 10 μg/mL final concentration.

The B3Z cell line, a hybridoma of CD8+ T-cells which is specific for OVA 257-264 residues in the context of MHC class I (H-2Kb), was grown in complete culture medium consisting of RPMI 1640 (GIBCO Cell Culture Systems) supplemented with 1% L-Alanyl-L-Glutamine (GlutaMAX ITM, GIBCO Cell Culture Systems), 10% fetal bovine serum (FBS) (Natocor S.A), 5×10-5 M 2β-mercaptoethanol (Sigma-Aldrich), 100 U/mL penicillin, and 100 μg/mL streptomycin (GIBCO Cell Culture Systems). The cells were incubated in an oven at 37° C. and controlled atmosphere with 5% CO2.

The cell line derived from hamster renal cells (BHK-21; ATCC CCL10) was used to amplify the FMDV virus. The cells were kept at 37° C. and 5% CO2 in Dulbecco's Modified Eagle medium (DMEM, Life Technologies, Grand Island, NY, USA) supplemented with 10% fetal bovine serum (FBS) and antibiotics (GibcoBRL/Invitrogen, Carlsbad, CA, USA).

### Example 2: Construction of Recombinant AcMNPV Baculoviruses

The “Bac to Bac” System (Invitrogen) was used to construct the recombinant AcMNPV, AcMNPV-OVAcap, AcMNPV-PCMVOVA, AcMNPV-VP1FMDVcap, AcMNPV-VP2FMDVcap, AcMNPV-Ag85TBcap, AcMNPV-ASP2TScap according to the specifications of the manual (O'Reilly, 1994).

For the construction, insert sequences were amplified and cloned into the pFBcap transfer vector (constructed and described previously in Molinari 20 11) under polyhedrin viral promoters using the primers shown in Table 1. The amplification products were initially cloned in pGemT and then subcloned in pFBcap by enzymatic restriction with XbaI and BamHI. The G insert product was cloned under the polyhedrin promoter in the pFB transfer plasmid. Construction of pFBPCMVOVA was carried out as described in Tavarone et al., 2018.

TABLE 1

| Primer sequences | | |
|---|---|---|
| used and | | |
| template.Primer | Primer sequence | Template |
| Gec for | ACCCGGGAAAGTTCACCATA | p-CMV VSV-G (Cell |
| | GTTTTTCC (SEQ ID No. 12) | Biolabs, San Diego, |
| Gec rev | GACTGCAGAAAAAATAAACTC | CA, EE. UU.) |
| | TCATCATC (SEQ ID No. 13) | |
| OVA for | AGGATCCAATATGCCTTTCAG | pRSET-OVA |
| | AGTGACT (SEQ ID No. 14) | |
| OVA rev | ATCTAGAAGGGGAAACACATC | |
| | TGCC (SEQ ID No. 15) | |
| Ag85ATB for | AGGATCCAATATGCAGCTTGT | pRSETAg85A |
| | TGAC (SEQ ID No. 16) | |
| Ag85ATB rev | ATCTAGACTAGGCGCCCTGG | |
| | GG (SEQ ID No. 17) | |
| VP1 FMDV for | ACTGCAGCGACCACCGCTAC | pRSETVP1 |
| | TGGGGAA (SEQ ID No. 18) | |
| VP1 FMDV rev | ACTGCAGCGCTGTTTTTCGGG | |
| | TGCAAT (SEQ ID No. 19) | |
| VP2 FMDV for | ACTCGAGAATATGGCCGACAA | pGEMTVP2 |
| | GAAGACGGAGGAG (SEQ ID | |
| | No. 20) | |
| VP2 MDV rev | ATCTAGACTCTTTCGAGGGGA | |
| | GCTC (SEQ ID No. 21) | |
| ASP2TS for | AAGATCTACCATGCTCGTGAA | pGEMTASP2TS |
| | CAGCCGGGAGGA (SEQ ID No. | |
| | 22) | |
| Gec for | ACCCGGGAAAGTTCACCATA | p-CMV VSV-G (Cell |
| | GTTTTTCC (SEQ ID No. 12) | Biolabs, San Diego, |
| Gec rev | GACTGCAGAAAAAATAAACTC | CA, EE. UU.) |
| | TCATCATC (SEQ ID No. 13) | |
| OVA for | AGGATCCAATATGCCTTTCAG | pRSET-OVA |
| | AGTGACT (SEQ ID No. 14) | |
| OVA rev | ATCTAGAAGGGGAAACACATC | |
| | TGCC (SEQ ID No. 15) | |
| Ag85ATB for | AGGATCCAATATGCAGCTTGT | pRSETAg85A |
| | TGAC (SEQ ID No. 16) | |
| Ag85ATB rev | ATCTAGACTAGGCGCCCTGG | |
| | GG (SEQ ID No. 17) | |
| VP1 FMDV for | ACTGCAGCGACCACCGCTAC | pRSETVP1 |
| | TGGGGAA (SEQ ID No. 18) | |
| VP1 FMDV rev | ACTGCAGCGCTGTTTTTCGGG | |
| | TGCAAT (SEQ ID No. 19) | |
| VP2 FMDV for | ACTCGAGAATATGGCCGACAA | pGEMTVP2 |
| | GAAGACGGAGGAG (SEQ ID | |
| | No. 20) | |
| VP2 MDV rev | ATCTAGACTCTTTCGAGGGGA | |
| | GCTC (SEQ ID No. 21) | |
| ASP2TS rev | ATCTAGAAAGAACCACGTGGT | |
| | ACCTAC (SEQ ID No. 23) | |

The recombinant constructions pFB or pFBcap or pFBPCMV were individually transformed into Escherichia coli DH10Bac cells (Invitrogen) to generate the corresponding recombinant bacmids, according to the manufacturer's instructions.

Briefly, 50 μL of E. coli competent DH10B were transformed with the transfer plasmids thus obtained. Selection of bacteria with the plasmid was performed in LB agar plates with 50 μg/mL kanamycin, 10 μg/mL tetracycline, and 7 μg/mL gentamycin; the transposition event was selected by the colony color (target phenotype) in the presence of 40 μg/mL IPTG and 100 μg/mL Bluo-gal. Bacmid purification was carried out as described in the manual of baculovirus expression (O'Reilly, 1994). The presence of the cassette of interest in the recombinant bacmids was then confirmed by PCR with M13 for universal specific oligonucleotides and M13 rev flanking the transposition site. For this reaction, the Taq DNA polymerase (Invitrogen) enzyme was used and the amplification cycle was of 95° C. 5 min, (94° C. 30 s, 55° C. 30 s, 72° C. 5 min)×30 cycles, 72° C. 7 min.

Recombinant baculovirus were obtained by the cell transfection method with cationic lipids. In a 1.5 ml tube, 100 UL of Sf 900 medium were mixed, with 2.5 μg of recombinant bacmid DNA and 100 μL of Sf900 medium with 10 μL of Cellfectina (Invitrogen). The mixture was incubated for 15-45 min at room temperature, 0.8 mL of Sf 900 medium were added and poured over a monolayer of Sf9 cells grown in six-well plates at a density of 1×106 cells/well. The plates were incubated for 4 h at 27° C., then the transfection medium was removed and 3 mL of Grace's medium supplemented with 10% FBS were added. After 6 days of incubation at 27° C., supernatants were collected in sterile form, clarified by centrifugation at 1,000×g for 10 min and preserved refrigerated. These recombinant baculoviruses were passaged two times in Sf9 cells by infecting 1×106 cells grown in 25 cm2 flasks with 100 UL of the transfection or infection supernatants. After 1 h of adsorption, 4 mL of Grace's culture medium supplemented with 10% FBS were added and the cells were incubated for 6 days at 27° C. Infection supernatants were collected in sterile form, clarified by centrifugation at 1,000×g for 10 min at 4° C. and kept refrigerated and protected from light. A third passage was performed in a 150 cm2 flask, which was used as viral stock. Baculovirus titration was carried out by the method of end point dilution (O'Reilly, 1994). Briefly, 300 μL of dilutions from 10-3 to 10-9 were added to the viral stocks at 2.7 mL Sf9 cells at a density of 2.5×105 cells/mL.

Then, 200 μL of each of these seven suspensions were placed in 12 wells of a 96 well plate, whereas in the remaining row non-infected cells were placed (negative control). Infected wells were recorded by GFP+fluorescence and viral titer was calculated by the Reed and Muench method.

### Example 3: Production of Recombinant AcMNPVs with Membranes Containing G Protein

Production of the recombinant baculoviruses containing in their membranes the vesicular stomatitis virus G protein (VSV) was carried out on one hand by generating a dual baculovirus (pFBdual) containing G under the P10 and OVAcap promoters and the Ppol promoter thereby obtaining AcMNPV-(G) OVAcap. The remaining viruses were obtained by co-infection of Sf9 cells with the AcMNPVG and AcMNPV-PCMVOVA or AcMNPV-Ag85ATBcap or AcMNPV-VP1FMDVcap or AcMNPV-VP2FMDVcap or AcMNPV-ASP2TSchagascap baculoviruses. Viral products of this infection contain G and the protein fused to cap.

In this way, the AcMNPV-(G) OVAcap, AcMNPV-(G) PCMVOVA, AcMNPV-(G) Ag85ATBcap, AcMNPV-(G) VP1FMDVcap and AcMNPV-(G) VP2FMDVcap, AcMNPV-(G) ASP2TSchagascap baculoviruses were obtained.

### Example 4: Presence of Antigens in Baculoviral Particles

### Western Blot

The presence of the antigens in baculoviruses was evaluated by purifying the viruses using ultracentrifugation on a sacarose pad (1.5 h at 80,000×g). Each simple was resuspended in cracking buffer and resolved by 12% SDS polyacrylamide gel electrophoresis and then the proteins were transferred onto nitrocellulose membranes.

The G protein was detected using a monoclonal anti-G antibody (clone P5D4, Sigma-Aldrich, Merck, Buenos Aires, Argentina, 1:1000) and mouse anti-goat IgG serum labelled with AP. For detection of the OVAcap protein, the rabbit anti-chicken ovoalbumin serum was used (1:5,000 manufactured in our laboratories) and rabbit anti-goat IgG serum labelled with AP (Sigma, 1:5000). The VP1FMDVcap and VP2FMDVcap proteins were detected with an anti-foot-and-mouth virus serotype A2001 rabbit antiserum and anti-rabbit IgG guinea pig antiserum labelled with AP. All these fusion proteins to VP39 were detected using a monoclonal anti-VP39 antibody and anti-mouse IgG goat antiserum labelled with AP.

### Example 5: Bone Marrow Dendritic Cells (DCs)

BMDCs were produced by growing bone marrow cells with the Fms-like tyrosine kinase 3 cytokine ligand in a similar way to that described by Brasel et al (2000). Supernatant of the SP2.0 cell line transfected with the gene encoding Flt3L was used as a source of Flt3L.

Bone marrow cells were obtained under sterile conditions from femurs, tibias and humerus of female C57BL/6 mice of 6 to 8 weeks of age. They were resuspended in complete culture medium and grown in culture plates for cells in suspension with 10% supernatant that contains Flt3L. On day 5, the same volume of complete medium with 10% supernatant rich in Flt3L was added. On day 9 or 10, BMDCs were obtained collecting both cells in suspension and those slightly adhered to the plate.

### Example 6: In vitro Antigenic Presentation Assay

To evaluate antigenic presentation in the context MHC class I of the stimulated DCs, the B3Z line, a CD8+ T-cell specific for the S8 peptide in the context of MHC I (H-2Kb) hybridoma, was used. Said hybridoma comprises the β-galactosidase enzyme under the control of the IL-2 promoter which allows for its activation.

BMDCs (2×105 cells per well) were incubated for 4 hours at 37° C. with different amounts of the AcMNPV-(G) OVAcap and AcMNPV-OVAcap baculoviruses in complete medium. BMDCs were incubated with 10 ng/ml OVA257-264 as a positive control. Then, cells were washed and co-incubated with 2×105 cells of the CD8+B3Z T hybridoma, for 18 hours at 37° C. in complete medium. Subsequently, supernatant was discarded and a colorimetric reaction was carried out to measure β-galactosidase activity using a lysis buffer containing chlorophenol red β-D-galactopyranoside (CPRG) (Roche Diagnostics Corporation), a substrate of this enzyme. After 4 hours of incubation at 37° C. in the dark, activity of the enzyme was quantified by measuring optic density at 595 nm in a plate reader.

### Example 7: In Vivo Cytotoxicity Assay

C57BL/6 mice of 6 to 8 weeks of age were i.v. immunized with different AcMNPV, AcMNPV-OVAcap, AcMNPV-(G) OVAcap, AcMNPV-PCMVOVA, AcMNPV-(G) PCMVOVA baculoviruses at different doses from 0.1×107 pfu and 2.5×107 pfu. On post-immunization day 7, an assay to evaluate cytotoxic T-lymphocyte generation was performed. Immunized animals were i.v. administered an equicellular mixture of syngenic splenocytes previously incubated with the 10 μg/mL OVA257-264 peptide and stained at a high concentration (3 μM) of Cell Proliferation Dye eFluor™ 670 (Invitrogen) (target cells) and normal splenocytes stained at a low concentration (0.3 μM) of Cell Proliferation Dye eFluor™ 670 (internal control). Spleens of the animals were obtained after twenty-four hours to evaluate the percentage of Cell Proliferation Dye eFluor™ 670-positive cells remaining therein by flow cytometry and the percentage of specific lysis was calculated. The following equation was used for calculating the percentage of specific lysis: (1−Rcontrol/Rimmunized)×100, where R=(% internal control cells)/(% target cells) for each evaluated mouse.

### Example 8: Lethal Challenge with Foot-and-Mouth Virus Assay

Quantification of FMDV viral particles was determined in BHK-21 cells by plaque assay (CFU/mL) or alternatively an infective dose of the 50% culture (TCID50) was calculated by the end point dilution method using the Reed and Muench formula (Reed and Muench, 1938).

Experimental animals were female C57BL/6J/LAE mice of 8 to 10 weeks of age from the biotherium of the Universidad Nacional de La Plata [National University of La Plata], Argentina. Mice were maintained under specific pathogen-free conditions and allowed an acclimatation period of 1 week at the animal biosafety 4 level facilities of the OIE (BSL-4 OIE) at the Instituto de Virología [Virology Institute], INTA. Experiments with the mice were performed according to the Institutional Animal Care and Use Committee (CICUAE-INTA protocol Nos. 15/2012 and 22/2013).

Groups of mice (n=4/5 per experimental group) of 6 to 8 weeks of age were i.v. immunized with 5×107 PFU of the AcMNPV or AcMNPV-VP1FMDVcap or AcMNPV-VP2FMDVcap baculoviruses and inoculated with 100 PFU FMDV A/Arg/01 by intraperitoneal injection 7 days after the last administered dose of immunogens. Post-challenge, mice were examined twice daily to record clinical symptoms of infection. Animals were euthanized when then showed irreversible signs of pain or disease (hypothermia, hunched posture, lethargy), according to a humanitarian endpoint.

### Example 9: Evaluation of the Response Against Mycobacterium tuberculosis Assay

Quantification of Mycobacterium tuberculosis strain H37Rv bacterium was performed by counting plaque-forming units. The animals were female Balb/c mice of 8 to 10 weeks of age from the biotherium of the Universidad Nacional de La Plata [National University of La Plata], Argentina. Mice were kept under specific pathogen-free conditions and allowed an acclimatation period of 1 week at the animal biosafety BSL-3 level facilities of the Instituto Nacional de Biotecnología [National Institute of Biotechnology], INTA. The animals were kept on ventilated racks.

Groups of mice (n=4/5 per experimental group) were i.v. immunized with different particles of AcMNPV85ATBcap or AcMNPV baculoviruses as controls (2×107 PFU/mL) and 7 days later they were intratraqueally challenged with 1×105 PFU under sedation). After 21 days, bacterial counts were analyzed in spleen and lung.

### Example 10: Evaluation of Protection Against Melanoma Assay

Groups of female C57BL/6J/LA mice of 6 weeks of age (n=6) were from the biotherium of the Universidad Nacional de La Plata [National University of La Plata], Argentina. The animals were kept under specific pathogen-free conditions, allowed a period of 1 week of acclimatation and then were i.v. immunized with AcMNPVOVAcap (5×107 PFU) or AcMNPV (5×107 PFU). After 7 days, the animals received implants by subcutaneous injection with 5×106 MO5 B16OVA tumor cells. The growth of tumor mass was recorded.

### REFERENCES

Blissard, G. W. (1996). Baculovirus—insect cell interactions. Cytotechnology, 20 (1-3), 73-93. doi: 10.1007/BF00350390
Brasel, K., De Smedt, T., Smith, J. L., & Maliszewski, C. R. (2000). Generation of murine dendritic cells from flt3-ligand-supplemented bone marrow cultures. Blood, 96 (9), 3029-3039.
Garry, C. E., & Garry, R. F. (2008). Proteomics computational analyses suggest that baculovirus GP64 superfamily proteins are class III penetrenes. Virol J, 5, 28. doi: 10.1186/1743-422X-5-28
Gheysen, D., Jacobs, E., of Foresta, F., Thiriart, C., Francotte, M., Thines, D., & De Wilde, M. (1989). Assembly and release of HIV-1 precursor Pr55gag virus-like particles from recombinant baculovirus-infected insect cells. Cell, 59 (1), 103-112. doi: 10.1016/0092-8674 (89) 90873-8
Kirnbauer, R., Taub, J., Greenstone, H., Roden, R., Durst, M., Gissmann, L., Schiller, J. T. (1993). Efficient self-assembly of human papillomavirus type 16 L1 and L1-L2 into virus-like particles. J Virol, 67 (12), 6929-6936. doi: 10.1128/JVI.67.12.6929-6936.1993
Latham, T., & Galarza, J. M. (2001). Formation of wild-type and chimeric influenza virus-like particles following simultaneous expression of only four structural proteins. J Virol, 75 (13), 6154-6165. doi: 10.1128/JVI.75.13.6154-6165.2001
Lj, R. (1938). A simple method of estimating fifty percent endpoints. Am J Hyg, 27, 493-495.
McMichael, A. J., Gotch, F. M., Noble, G. R., & Beare, P. A. (1983). Cytotoxic T-cell immunity to influenza. N Engl J Med, 309 (1), 13-17 . . . doi: 10.1056/NEJM198307073090103
Molinari, P., Crespo, M. I., Gravisaco, M. J., Taboga, O., & Moron, G. (2011). Baculovirus capsid display potentiates OVA cytotoxic and innate immune responses. PLOS One, 6 (8), e24108. doi: 10.1371/journal.pone.0024108
Molinari, P., Molina, G. N., Tavarone, E., Del Medico Zajac, M. P., Moron, G., & Taboga, O. (2018). Baculovirus capsid display in vaccination schemes: effect of a previous immunity against the vector on the cytotoxic response to delivered antigens. Appl Microbiol Biotechnol, 102 (23), 10139-10146. doi: 10.1007/s00253-018-9368-8
O'Reilly, D. R., Miller, L. K., & Luckow, V. A. (1994). Baculovirus Expression Vectors: A Laboratory Manual: Oxford University Press.
Tavarone, E., Molina, G. N., Amalfi, S., Peralta, A., Molinari, P., & Taboga, O. (2017). The localization of a heterologous displayed antigen in the baculovirus-budded virion determines the type and strength of induced adaptive immune response. Appl Microbiol Biotechnol, 101 (10), 4175-4184. doi: 10.1007/s00253-017-8183-and
Yoshida, S., Kawasaki, M., Hariguchi, N., Hirota, K., & Matsumoto, M. (2009). A baculovirus dual expression system-based malaria vaccine induces strong protection against Plasmodium berghei sporozoite challenge in mice. Infect Immun, 77 (5), 1782-1789. doi: 10.1128/IAI.01226-08

1. A recombinant baculovirus, characterized by comprising a) a nucleotide sequence encoding a fusion protein, wherein said fusion protein consists of an antigen fused to a baculovirus capsid peptide operatively linked to a first promoter and b) a nucleotide sequence encoding a lipid viral envelope protein bound to a second promoter.

2. The baculovirus according to claim 1, characterized in that the antigen is an antigen selected from the group consisting of viral antigens, bacterial antigens, tumor antigens, parasite antigens, multi-antigens and fragments thereof.

3. The baculovirus according to claim 1, characterized in that the baculovirus capsid protein is selected from the group consisting of VP39, GP64, p6.9, AC104, AC144, AC101, AC109, AC142, and Ac98.

4. The baculovirus according to claim 1, characterized in that the nucleotide sequence encoding the peptide is VP39 comprising the nucleotide sequence set forth in SEQ ID No. 6.

5. The baculovirus according to claim 1, characterized in that the antigen is selected from the group consisting of the OVA 257-264 epitope, MO5 epitope, Mycobacterium tuberculosis Ag85A, foot-and-mouth virus VP1, foot-and-mouth virus VP2, and Trypanosoma cruzi ASP2 fused to Trypanosoma cruzi TS.

6. The baculovirus according to claim 1, characterized in that the first and second promoter is an insect cell promoter.

7. The baculovirus according to claim 6, characterized in that the first promoter is selected from the group consisting of Ppol, P10, Pie1, PDUO, and PTRIX.

8. The baculovirus according to claim 6, characterized in that the second promoter is selected from the group consisting of Ppol, P10, Pie1, PDUO, and PTRIX.

9. The baculovirus according to claim 1, characterized in that the second promoter is selected from the group consisting of PCMV, PCAG, PTRIX, and PDUO.

10. The baculovirus according to claim 1, characterized in that the antigen is the MO5 epitope.

11. The baculovirus according to claim 1, characterized in that the antigen is Mycobacterium tuberculosis Ag85A and is encoded by the nucleotide sequence of SEQ ID No. 2

12. The baculovirus according to claim 1, characterized in that the antigen is the foot-and-mouth virus VP1 and is encoded by the nucleotide sequence of SEQ ID No. 4.

13. The baculovirus according to claim 1, characterized in that the antigen is the foot-and-mouth virus VP2 and is encoded by the nucleotide sequence of SEQ ID No. 5.

14. The baculovirus according to claim 1, characterized in that the antigen is Trypanosoma cruzi ASP2 fused to Trypanosoma cruzi TS and is encoded by the nucleotide sequence of SEQ ID No. 3.

15. The baculovirus according to claim 1, characterized in that the sequence of the lipid viral envelope protein is selected from the group consisting of the vesicular stomatitis virus (VSV) G protein, rhabdovirus G protein, togavirus GP and herpesvirus gB.

16. The baculovirus according to claim 15, characterized in that the lipid viral envelope protein is the vesicular stomatitis virus G protein and is encoded by the nucleotide sequence set forth in SEQ ID No. 6.

17. The baculovirus according to claim 1, characterized in that it expresses a fusion protein selected from the group consisting of the amino acid sequences of SEQ ID No. 7, SEQ ID No.8, SEQ ID No. 9, SEQ ID No. 10, and SEQ ID No. 11.

18. A recombinant baculovirus, characterized by comprising a nucleotide sequence encoding a fusion protein, wherein said fusion protein consists of an antigen fused to a baculovirus capsid peptide operatively linked to a promoter, wherein the fusion protein has an amino acid sequence selected from the group consisting of SEQ ID No. 7, SEQ ID No. 9, SEQ ID No. 10, SEQ ID No. 11, and SEQ ID No. 12, and wherein the fusion protein is expressed on the capsid.

19. The baculovirus according to claim 18, characterized in that the promoter is selected from the group consisting of Ppol, P10, and Piel.

20. A recombinant baculovirus, characterized by comprising a nucleotide sequence encoding the vesicular stomatitis virus G protein operatively linked to a promoter, wherein the G protein is encoded by the nucleotide sequence of SEQ ID No. 6.

21. The baculovirus according to claim 20, characterized in that the promoter is selected from the group consisting of Ppol, P10, and Piel.

22. The baculovirus according to claim 20, characterized in that the promoter is selected from the group consisting of PCMV, PCMV, PSV40, and PTriEx.

23. A vaccine composition, characterized by comprising the recombinant baculovirus of claim 1.

24. A vaccine composition, characterized by comprising the recombinant baculovirus of claim 18 and the baculovirus of claim 20.

25. A method for inducing an immune response, characterized by comprising administering to a subject a pharmaceutically effective amount of the recombinant baculovirus of claim 1.

26. A method for inducing an immune response, characterized by comprising concurrently administering to a subject a pharmaceutically effective amount of the recombinant baculovirus of claim 18 and the recombinant baculovirus of claim 20.

27. The method for inducing an immune response according to claim 25, characterized in that an amount from 1×106 to 2,7×108 CFU of the baculovirus is applied.

28. The method for inducing an immune response according to claim 26, characterized in that an amount from 1×106 to 2,7×108 CFU of each baculovirus is applied.
